(19) United States
(12) Patent Application Publication
(10) Pub. No.: US 2007/0269495 A1
(43) Pub. Date: Nov. 22, 2007

(54) Compositions and methods for enhancing mineral levels in animals with reduced environmental impact

(76) Inventor: H. DeWayne Ashmead, Fruit Heights, UT (US)

Correspondence Address: THORPE NORTH & WESTERN, LLP., 8180 SOUTH 700 EAST, SUITE 350, SANDY, UT, 84070, US

(21) Appl. No.: 11/437,871

(22) Filed: May 18, 2006

(51) Int. Cl. A61K 31/555; A23K 1/165; A61K 31/28

(52) U.S. Cl. 424/442; 514/492; 514/184

(57) ABSTRACT

A method of enhancing a mineral level in an animal with reduced environmental impact can comprise orally administering a metal amino acid chelate to an animal, wherein the amino acid to metal molar ratio of the metal amino acid chelate is from about 1:1 to 4:1. The metal can contribute to a mineral level within the blood and tissues of the animal that is effective for stimulating growth of the animal to a greater degree than would be realized by administering the same amount of metal in the form of an inorganic metal salt. Additionally, the amount of the metal excreted in the feces of the animal can be less than would be present when administering the same amount of metal in the form of the inorganic metal salt. The metal amino acid chelate can also serve as a source highly bioavailable amino acids, so that the excretion of nitrates by the animal may be reduced while still promoting efficient growth.

### FIELD OF THE INVENTION

The present invention relates to compositions and methods for promoting growth in animals, such as livestock animals, by supplementing their diets with essential minerals. More particularly, the present invention is directed to enhancing the absorption of ingested minerals in these animals while at the same time reducing the fecal mineral levels from animals.

### BACKGROUND OF THE INVENTION

A significant portion of human agricultural efforts is devoted to the raising and breeding of livestock such as cattle, swine, or poultry. Millions of animals worldwide are kept in livestock operations for their meat, eggs, milk, hides, fur, or as breeding stock. Not surprisingly, such an immense number of animals produce tremendous amounts of waste. For example, a single pig may produce as much as 7.1 kilograms of waste per day. In the United States, for example, livestock produced roughly one trillion pounds of waste in 1997 alone. The handling and disposition of so much waste is therefore a principal concern of individual farmers, localities, and nations as a whole.

In many types of livestock production operations, particularly those where livestock are kept in confinement, animal waste must be actively managed. In these cases, the waste is commonly collected from the animals' living space and gathered at a designated location. For example, large-scale swine production facilities often utilize large “lagoons” in which to collect liquid and solid wastes from the animals. Gathering waste in these ways has the effect of concentrating the waste, as well as the odors, ammonia, and pathogens that may accompany it. Nitrogen, phosphorus, and heavy metals from animal feces may accumulate in the adjacent soil, disrupting the nutrient balance and decreasing its arability. Nitrates in the soil may in turn be carried to local streams and rivers via runoff, fostering excessive algal growth and increasing fish mortality. Therefore, waste accumulations associated with livestock operations have the potential to adversely affect surrounding air, soil, and water.

The particular composition of livestock waste is often a direct product of specialized diets fed to the animals in order to increase yield of salable product. Normal growth in livestock animals requires a diet that includes sufficient amounts of essential trace metals such as copper, phosphorus, zinc, and manganese, to name a few. Faster growth can often be achieved by providing the animals with amounts of vitamins and minerals in excess of minimum requirements. Typically this is done by supplementing the animals' diet with inorganic metal salts. However, ingested inorganic metals are often absorbed in the digestive tract with poor efficiency, due to the saturability of the mechanisms that transport these ions out of the intestinal lumen. Therefore, as much as 85% of ingested inorganic metal is typically excreted in its feces and/or urine.

Livestock waste pollution is a growing concern among governments worldwide, as reflected in more restrictive regulations aimed at decreasing the accumulation of minerals in soil. Livestock producers in these places are therefore confronted with the choice of decreasing their yields or violating anti-pollution regulations.

### SUMMARY OF THE INVENTION

It has been recognized that it would be advantageous to develop a method for enhancing the amount of metal absorbed into the blood and tissue of an animal while reducing the amount of the metal present in the feces of the animal. In accordance with this, a method of enhancing a mineral level in an animal with reduced environmental impact can comprise orally administering a metal amino acid chelate to an animal, wherein the amino acid to metal molar ratio of the metal amino acid chelate is from about 1:1 to 4:1. The metal can contribute to a mineral level within the blood and tissues of the animal that is effective for stimulating growth of the animal to a greater degree than would be realized by administering the same amount of metal in the form of an inorganic metal salt. Further, the amount of the metal excreted in the feces of the animal can be less than would be present when administering the same amount of metal in the form of the inorganic metal salt. Administering the metal amino acid chelate can also result in excretion of a lower amount of nitrates than would result from ingesting an equal amount of amino acid from other sources. A further step can comprise reducing the animal's protein intake from other sources without reducing its growth rate.

In another embodiment, a method for facilitating the recovery of animal feces byproducts by reducing the amount of a metal in the feces byproducts can comprise orally administering a metal in the form of a metal amino acid chelate to an animal, wherein the amount of the metal administered is sufficient for stimulating growth of the animal while resulting in a lower amount of the metal in the feces of the animal than would result from administering the same amount of the metal in an inorganic salt form. The amount of nitrates present in the feces can also be less than would be present if the animal had ingested an equal amount of amino acid from other dietary sources. A further step can include recovering the feces from the animal, wherein the feces has a low enough concentration of metal to be acceptable for soil enrichment.

In another embodiment, a feed that provides a metal to an animal in a highly bioavailable form can comprise one or more metal amino acid chelates, where the amount of metal the animal ingests by eating the feed contributes to stimulating growth of the animal to a greater degree than the same amount of metal is ingested by the animal in the form of an inorganic metal salt. Further, the amount of metal excreted in the feces of the animal is less than would be present when the same amount of metal is ingested by the animal in the form of the inorganic metal salt.

Additional features and advantages of the invention will be apparent from the detailed description which follows, taken in conjunction with the accompanying drawings, which together illustrate, by way of example, features of the invention.

### DETAILED DESCRIPTION OF THE PREFERRED EMBODIMENT(S)

Reference will now be made to certain exemplary embodiments of the invention, and specific language will be used herein to describe the same. It will nevertheless be understood that no limitation of the scope of the invention is thereby intended. Alterations and further modifications of the inventive features illustrated herein, and additional applications of the principles of the inventions as illustrated herein, which would occur to one skilled in the relevant art and having possession of this disclosure, are to be considered within the scope of the invention.

It is to be noted that, as used in this specification and the appended claims, the singular forms “a,” “an,” and “the” include plural referents unless the context clearly dictates otherwise.

The terms “metal” and “mineral” may be used interchangeably. Each refers particularly to any divalent or trivalent metal that, when in ionic form, can form one or more coordinate bonds with a ligand, and is substantially non-toxic when administered in traditional amounts as known in the art. The metal is preferably a metal selected from the group consisting of Cu, Mn, Mg, Fe, Zn, Cr, and Ca.

The term “amino acids” or “naturally occurring amino acids” shall mean α-amino acids that are known to be used for forming the basic constituents of proteins, including alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, valine, and combinations thereof.

When referring to “metal salts” or “salts,” it is recognized that oxides and hydroxides are not technically salts in the classic sense. However, in accordance with embodiments of the present invention, metal oxides and hydroxides are considered to be salts along with any other metal salts as more typically defined.

Reference is made to a number of molecular arrangements in which a metal ion is bonded to one or more molecules. The terms “chelate,” “metal amino acid chelate,” or the like can be used interchangeably herein, and refer to a product resulting from the reaction of one or more amino acid ligands with a metal ion at a molar ratio of 1:1 to 4:1, and typically 1:1 to 3:1. In one preferred embodiment, the molar ratio is about 2:1. Each amino acid of the chelate bonds to the metal both at the α-amino nitrogen and the carboxyl oxygen of the amino acid to form a ring structure. Therefore, each chelate features one or more five-member heterocyclic rings, each ring comprising the metal atom, as well as an amino acid's α-amino nitrogen, α-carbon, carbonyl carbon, and carboxyl oxygen. The bond formed by the α-amino nitrogen is typically a coordinate bond, where both electrons of the bond are donated by the nitrogen. The bond formed by the carboxyl oxygen may be coordinate, covalent, or ionic, though preferably it is a coordinate bond. In every case, the use of the term “chelate” requires that a ring structure be formed which includes both the amino acid ligand and the metal.

As used herein, the term “livestock” includes warm-blooded animals kept or raised for use or pleasure. Typically, “livestock” refers to animals that are commonly kept or raised for some commercial use or purpose. These may be animals that are kept in confinement within a building or shelter, or within some partially or fully enclosed area of land. Alternatively, livestock may be allowed to roam freely over an open area of land. In one specific embodiment, “livestock” refers to animals selected from the group consisting of swine, ruminants, poultry, equines, and any combination thereof.

As used herein, in reference to livestock, the term “growth” may refer to an increase in the height, length, width, or mass of the animal's own body, or any combination of these measurements. Such increases may occur in an animal that has reached sexual maturity as well as in developing animals that are still sexually immature.

The term “supplement” as used herein shall mean any foodstuff, composition, or compound that contains a substance intended to benefit an animal, and is provided to the animal in order to increase the amount of that substance ingested by the animal above the amount it receives by its normal dietary behavior.

The term “orally administer” means delivering a compound or composition to a living animal so that the compound, composition, or a component thereof may be taken in orally and ingested by the animal. Some typical methods of administering substances to livestock animals include presenting the animal with a compound or composition in an edible form either alone or with the animal's feed or water, or injecting a liquid or a bolus of solid directly into the animal's mouth.

It is well known that the growth and development of livestock animals is promoted by the presence of adequate amounts of essential vitamins and minerals in their diet. Minerals that are considered desirable for animal growth include phosphorus, copper, manganese, and zinc. Other valuable minerals for many livestock include iron, calcium, and magnesium. These minerals are often present in trace amounts in animal forage or in plant-based components of commercially available feed. However, the amount of metal present in these foods may be small or highly variable in metal content. One may therefore supplement the diet of an animal with an essential metal, so that the animal receives a greater amount of metal than it typically would from the forage and feed materials it eats. Typically metals are administered to livestock animals in the form of inorganic metal salts, such as metal oxides, metal hydroxides, metal sulfates, metal phosphates, metal carbonates, and/or metal chlorides. Metals are also commonly administered to livestock in the form of other metal complexes, such as metal complex proteinates, metal propionates, and yeast derivative complexes. Supplementation of this kind has been shown to increase growth rates in livestock animals. However, the efficiency with which ingested inorganic metals are absorbed into the blood is quite low, and thus, the animal usually excretes much of the ingested metal in its feces and urine. Therefore, while supplementing the diet of livestock contributes to more rapid animal growth, it also results in the production of waste having a high metal content. Accumulation of such waste, particularly feces waste, can exert a negative impact on the environment, due to large amounts of metal leaching into the soil and nearby water.

A method for enhancing a blood mineral level so as to promote faster growth in an animal with reduced environmental impact can comprise orally administering metal amino acid chelates to the animal. In particular aspects of this invention, metal amino acid chelates, e.g., copper, zinc, or manganese, may be administered to the animal. However, it will be appreciated by those having skill in the art that, in accordance with this invention, one may administer other nutritionally relevant metals that can bind with an amino acid to form a chelate, e.g., iron, calcium, magnesium, chromium, etc. Members of the group of naturally occurring amino acids can be used as the ligand(s), binding to the metal to be administered via coordinate, covalent, or ionic bonds. Metals having a sufficient number of free coordination sites may in fact bind one, two, or three amino acid molecules (and sometimes four). For any given metal, the same amino acid may constitute all of its ligands, or any combination of different amino acids may also bind to a particular metal ion. It will be apparent to those skilled in the art that a large number of metal/amino acid combinations are in accordance with embodiments of the present invention. It is also to be understood that the present invention is intended to encompass all possible combinations that include naturally occurring amino acids chelated to metals which may be administered to an animal in order to promote growth.

Different practices exist in the art for the keeping of livestock, each depending on the kind of animal kept, the size of the livestock operation, and the amount of space available. Some livestock farming methods involve keeping livestock animals confined in buildings or other restricted spaces. Confined livestock animals may obtain their feed from troughs or feed dispensers, or their feed may be scattered on the floor or ground of their confinement area. Under other practices, livestock animals are allowed to roam freely over a wide area of land, and may obtain most of their food from foraging. Accordingly, different embodiments of the present invention may be employed in administering chelates to animals.

A metal amino acid chelate may be administered to an animal in accordance with this invention by combining the chelate with the animal's feed. In a particular embodiment, the chelate may be intermixed with the feed before presenting the mixture to the animal. In another embodiment, a chelate and other optional additives can be added to the feed during processing and then administered to the animal as a fortified feed. In another embodiment, the chelate may be shaped (with a foodstuff or a carrier) into a pellet or bolus and delivered directly into the animal's mouth with a bolusing gun. In yet another embodiment, a chelate may be dissolved or suspended in the drinking water provided to the animal. In still another embodiment, a chelate may be dissolved or suspended in a potable liquid. A common way known in the art for administering such a liquid is to deliver it directly into the mouth of the animal with a drench gun.

A chelate may be also administered to free-roaming animals in accordance with this invention by mixing the chelate with feed or liquid and placing the feed or liquid at one or more locations within the animals' foraging range. Alternatively, the chelate may be included in a solid composition. The composition may be provided in a cube, block, or tub, or in a feeder and then placed at one or more locations within the animal's foraging range.

As discussed, livestock animals can produce tremendous amounts of liquid and solid waste. Often the sheer volume of this waste makes it necessary to collect it, remove it from the animal living area, and confine it to a space designated for this purpose. Collected waste may produce odorous gases that affect air quality, thereby limiting the usability of surrounding land. Semi-liquid waste such as is typically collected in waste lagoons can provides a breeding site for mosquitoes and other pests. One approach to managing animal waste is to recover the waste and adapt it for use as a fertilizer for soil enrichment. This is often accomplished by collecting waste in composting piles or waste lagoons, and allowing bacterial action to decompose the manure solids, converting them into stable organic matter that contain nutrients that promote plant growth. However, high levels of metals in feces may limit its use as a fertilizer. While crop plants benefit from trace metals in the soil in order to grow properly, they can be present in very small amounts. For example, without being so limited, soil levels of 40-50 ppm Mg, 1.0 ppm Mn, 0.2 ppm Cu, and 1.0 ppm Zn are considered adequate for most crop types. On the other hand, levels slightly above that required for growth can be toxic to crop plants. This is particularly true for copper and zinc, which can stunt plant growth at levels of 60 and 120 ppm, respectively. Most soil already contains significant amounts of trace metal, so for a manure-based fertilizer to be usable, it should not increase these levels greatly. In light of these recognitions, the present invention also provides a method for facilitating recovery of animal feces, where the feces has a lower content of the metal, and therefore, is more suitable for use in soil enrichment. In a particular embodiment, the feces may be substantially free of the metal. Once collected, the animal feces can added to the soil.

Supplementing the diets of livestock animals with minerals is highly recommended to promote normal growth. However, using inorganic metal salts as a supplement provides these metals in a form that is not easily utilized by the animals, because the limited population of metal transporter molecules in the intestine only allows for absorption of a portion of ingested metal ions. In light of these recognitions, the present invention also encompasses a feed composition that includes metal amino acid chelates, and may be fed to a livestock animal so as to provide a metal to the animal in a highly bioavailable form. The metal amino acid chelates provided by the present invention can be absorbed rapidly and efficiently in the intestine. Therefore, by feeding an animal a feed composition in accordance with this invention, more of the metal it ingests can be absorbed into the bloodstream. As a result, the animal will attain a higher blood level of the metal than it would from ingesting the same number of moles of metal in an inorganic metal salt. The animal will also excrete less of the metal in its feces.

Such a feed may further comprise any grain or mixture of grains that are edible by livestock animals, such as corn, wheat, rye, barley, sorghum, oats, rice, cottonseed meal, and canola, as well as other foodstuffs such as soybeans, milk products, meat, bone meal, feather meal, and byproducts from food processing. The feed may further comprise an additive or mixture of additives selected from the group consisting of vitamins, flavor enhancers, aroma enhancers, colorings, fiber, yeast, ground limestone, potassium chloride, stabilizers, emulsifiers, sequestrants, preservatives, anti-oxidants, and anti-caking agents. It is notable that the amino acid chelates can be blended with foods, incorporated into foods, suspended in feed liquids, or dissolved in feed liquids.

A diet with adequate amounts of protein is also significant to the health and growth of livestock animals. A result of the ingestion and digestion of proteins is the production of nitrates, which are excreted in the waste of the animals. As briefly mentioned above, nitrates in large amounts of animal waste can adversely affect the environment, in part because excess soil nitrates can run off into streams and rivers. Feeding amino acid chelates to animals in accordance with the present invention can, in addition to providing essential dietary minerals, serve as a dietary source of amino acids. Furthermore, providing amino acids in this form can result in more amino acids being available to the animal for digestion when compared to feeding with protein. Therefore, feeding an animal amino acid chelates allows for one to decrease the amount of protein in the animal's diet while still achieving effective metabolic response. An additional result is that the animal will excrete a lower amount nitrate in its waste compared to an animal on a conventional diet. In light of these recognitions, the methods of the present invention provide a way to decrease the nitrate content of animal waste by administering metal amino acid chelates to the animal. The higher bioavailability of the amino acids in the chelates provide a protein sparing effect, so that these amino acids can replace those that the animal would usually obtain from other dietary sources. A nutritionist skilled in the art could reformulate animal feed that contains less protein than conventional feeds and that, when fed to animals in conjunction with administration of metal amino acid chelates, produces the similar growth and production from the animal as conventional feed while reducing the amount of nitrates in its waste.

### EXAMPLES

### Effects of Administering Metal Amino Acid Chelates on Metal Content in the Waste of Livestock Animals

In Examples 1-3 below, pregnant Lanebrace X Yorkshire crossbred sows were housed in a barn having a thermostat-controlled average temperature of 18.9° C. to 20° C. The sows were isolated in full-length gestation crates (length=2.03 m, height=0.99 m, width=0.61 m). The crates were situated on partial slatting to allow easy removal of feces from the 0.91 m manure pit below.

A basal feed ration was prepared by adding manganese oxide, zinc oxide, and copper sulfate to feedstuff to supplement the natural amount of these metals therein. To this basal feed was added an additional amount of one metal, either as a metal amino acid chelate (AAC) or as an inorganic metal salt (IM). For three weeks, each sow was hand-fed a 2 kg ration per day, either of the AAC feed or of the IM feed containing the metal supplement designated for the sow's group. The sows were fed once per day and would eat the complete meal in about 10 minutes. Water was provided ad libitum for 23.5 hours each day.

After the three-week period, the daily defecation of each sow was directly collected into separate plastic containers, which were then sealed, double-bagged, and sent to an independent analytical laboratory for analysis. The manure samples were individually digested in perchloric, nitric, and hydrochloric acids for full digestion of minerals, including any that may have been chelated. Quantitative analyses of metal content were accomplished by inductive-coupled plasma spectrometry (ICP). Between-group comparisons of mean fecal metal content were made using Cochran's t-prime test.

### Example 1

### Administration of Copper Chelates

A group of 32 sows were fed a daily ration of feed containing 27.5 ppm Cu, 9.09% which was in AAC form. Another 35 sows were fed a daily ration of feed containing 27.5 ppm Cu, all in the form of copper sulfate. Each ration provided a total of 59.7 mg of supplemental Cu per day to each sow. The amounts of Cu found in the manures expressed on a dry weight basis is shown in Table 1 below.

TABLE 1

| | Amount of | Mean ± SD | |
|---|---|---|---|
| Type of | supplemental | metal content | |
| supplement | metal received | in feces | p-value of t′ |
| IM | 59.7 mg/day | 310 ± 39 ppm | p < 0.0005 |
| | (100% as CuSO₄) | | |
| AAC | 59.7 mg/day | 271 ± 25 ppm | |
| | (9.09% as chelate) | | |

### A t-prime analysis of this data shows that the metal content in the feces of sows receiving part of their dietary copper from chelates was significantly lower than in the feces of sows that received all of their copper from inorganic salts.

### Example 2

### Administration of Manganese Chelates

A group of 29 sows were fed a daily ration of feed containing 59.9 ppm Mn, 33.3% which was in AAC form. This ration provided each sow with 119.4 mg of Mn per day. A second group of 32 sows were fed a daily ration of feed containing a higher proportion of Mn (71.4 ppm), 44.12% of which was in the form of manganese sulfate (the remainder from manganese oxide). This ration provided each pig in this group with 142.4 mg Mn per day. The amounts of Mn found in the manures expressed on a dry weight basis is shown in Table 2 below.

TABLE 2

| | Amount of | Mean ± SD | |
|---|---|---|---|
| Type of | supplemental | metal content | |
| supplement | metal received | in feces | p-value of t′ |
| IM | 142.4 mg/day | 920 ± 305 ppm | p < 0.0005 |
| | (44.12% as MnSO₄) | | |
| AAC | 119.4 mg/day | 628 ± 80 ppm* | |
| | (33.3% as chelate) | | |

*In order to adjust for the fact that the sows on the IM diet received a 119.2% higher proportion of Mn in their feed, the individual manure assays from the sows receiving the AAC diet were multiplied by 1.1920 to yield the adjusted value shown.

### A t-prime analysis of this data shows that the metal content in the feces of sows receiving part of their dietary manganese from chelates was significantly lower than in the feces of sows that received all of their manganese from inorganic salts.

### Example 3

### Administration of Zinc Chelates

A group of 32 sows were fed a daily ration of feed containing 392.2 ppm of Zn, 12.72% of which was in the AAC form. The ration fed to a second group of sows contained 393.9 ppm of Zn, 12.81% of which was in the sulfate form (the remainder was from zinc oxide). These diets yielded similar daily amounts of total Zn, 852.6 mg and 853.5 mg, respectively. The amount of Zn found in each of the manures expressed on a dry weight basis is shown in Table 3 below.

TABLE 3

| | Amount of | Mean ± SD | |
|---|---|---|---|
| Type of | supplemental | metal content | |
| supplement | metal received | in feces | p-value of t′ |
| IM | 852.6 mg/day | 3951 ± 490 ppm | p < 0.05 |
| | (12.81% as ZnSO₄) | | |
| AAC | 853.5 mg/day | 3770 ± 319 ppm | |
| | (12.72% as chelate) | | |

### A t-prime analysis of this data shows that the metal content in the feces of sows receiving part of their dietary zinc from chelates was significantly lower than in the feces of sows that received all of their zinc from inorganic salts.

### Effects of Administering Metal Amino Acid Chelates on Amino Acid Availability in Various Livestock Animals

### Example 4

### Poultry

A group of 180 male broilers were divided into groups of 30 birds each. Half were control birds and half were treated birds. The treated groups were given one of the supplements of metal amino acid chelates shown in Table 4 below.

TABLE 4
Composition of Amino Acid Chelate Supplements Fed to Poultry

| | % in Mineral | % in Feed at Different Levels | | |
|---|---|---|---|---|
| Mineral | Mix Formula | 50 ppm | 100 ppm | 200 ppm |
| Iron | 9.00% | 0.0045% | 0.009% | 0.018% |
| Copper | 2.15% | 0.00107% | 0.00215% | 0.0043% |
| Zinc | 3.00% | 0.0015% | 0.0030% | 0.0060% |
| Manganese | 1.20% | 0.0006% | 0.0012% | 0.0024% |
| Cobalt | 0.08% | 0.00004% | 0.00008% | 0.0002% |

The control birds did not receive supplements. Three diets were fed. The principle source of protein was derived from corn (maize), soybean, or barley, as seen in Table 5 below.

TABLE 5
Experimental Design for Broilers

| | Number of | Supplement | | |
|---|---|---|---|---|
| Group Feed | Birds | 50 ppm | 100 ppm | 200 ppm |
| Control corn | 30 | — | — | — |
| Experimental corn | — | 10 | 10 | 10 |
| Control soybean | 30 | — | — | — |
| Experimental | — | 10 | 10 | 10 |
| soybean | | | | |
| Control barley | 30 | — | — | — |
| Experimental barley | — | 10 | 10 | 10 |

The feed also contained Cr₂O₃ which was used as an indication digestibility of the protein in the feed and as proportionality constant to measure the amino acid content of the feeds. The birds received the fortified feeds for 60 days at which time they were sacrificed. The ileocecal tract was removed and feces, not contaminated with urine, were collected. Amino acid content was measured by a Beckman automatic analyzer using acid-insoluble ash and Cr₂O₃ as markers. Table 6 shows the results.

TABLE 6
Percentage Increases of Metabolizable Available Amino Acid form Broiler
Feeds Containing Amino Acid Chelates in Birds Receiving Amino Acid
Chelates Compared to Control Birds

| Amino Acid | Corn Diet % | Soybean Diet % | Barley Diet % |
|---|---|---|---|
| Aspartic acid | +2.8 | +0.7 | +3.1* |
| Threonine | +1.7 | +1.2 | +4.3* |
| Serine | +1.3 | +0.4 | +2.6 |
| Glutamic acid | +8.8* | +4.3* | +9.8* |
| Proline | +7.1* | +6.4* | +8.1* |
| Alanine | +3.4* | +1.1 | +2.5 |
| Valine | +3.1 | +4.3* | +8.2* |
| Methionine | +3.7* | +3.2* | +7.6* |
| Isoleucine | +1.6 | +1.6 | +2.1 |
| Leucine | +10.4* | +6.3* | +12.7* |
| Tyrosine | +7.3* | +3.9* | +11.1* |
| Phenylalanine | +5.9* | +2.5 | +2.8 |
| Lysine | +10.8* | +7.7* | +12.3* |
| Histidine | +3.9* | +2.4 | +5.7* |
| Arginine | +2.3 | +0.8 | +3.4 |

*p < 0.05

There was an increase in the amount of digestible amino acids from the diets of the birds receiving the chelates. Many of these increases were significant.

### Example 5

### Swine

A group of 36 male pigs (28 days old at study commencement) were divided into 2 groups: experimental and control. The experimental group was further divided into 3 groups. The pigs were fed a weaner feed for 30 days, after which time the feed was switched to a grower feed. All pigs in both groups received the same feed. Each of the experimental group also received a supplement of metal amino acid chelates shown in Table 7.

TABLE 7
Composition of Amino Acid Chelate Supplements fed to Swine

| | % in Mineral | % in Feed at Different Levels | | |
|---|---|---|---|---|
| Mineral | Mix Formula | 50 ppm | 100 ppm | 200 ppm |
| Iron | 9.00% | 0.0045% | 0.009% | 0.0180% |
| Copper | 2.15% | 0.00107% | 0.00215% | 0.0043% |
| Zinc | 3.00% | 0.0015% | 0.0030% | 0.0060% |
| Manganese | 1.20% | 0.0006% | 0.0012% | 0.0024% |
| Cobalt | 0.08% | 0.00004% | 0.00008% | 0.0002% |

Each pig in the experimental group was fed chelate supplements at 50 ppm, 100 ppm, or 200 ppm for 60 days. A Cr₂O₃ marker was included in the feed as described in Example 4. At the end of 30 days when the feed was changed and again at 60 days when the study was terminated fecal samples were obtained and assayed for available amino acid contents using the method described in Example 4. As shown in Table 8, almost every amino acid from the protein fed to the swine was more available when amino acid chelates were included in the feed.

TABLE 8
Percentage Increase of Available Amino Acids Form Swine Feeds in Pigs
Receiving Amino Acids Chelates Compared to Control Pigs

| | First 30 days | Second 30 days |
|---|---|---|
| Amino Acid | Weaner Feed % | Grower Feed % |
| Aspartic acid | +3.8* | +1.8* |
| Threonine | +0.6 | +0.2 |
| Serine | +5.7* | +4.4* |
| Glutamic acid | +3.2* | +2.3 |
| Proline | +8.9* | +5.3* |
| Alanine | +0.8 | +0.2 |
| Valine | +4.5* | +4.0* |
| Methionine | +7.7* | +6.1* |
| Isoleucine | +0.2 | 0 |
| Leucine | +8.9* | +4.7* |
| Tyrosine | +5.9* | +5.4* |
| Phenylalanine | +5.3* | +4.2* |
| Lysine | +8.8* | +6.2* |
| Histidine | +4.5* | +3.3 |
| Arginine | +0.3 | −0.4 |

*p < 0.05

### Example 6

### Cattle

A group of 40 (10 week old) male calves were divided into 2 groups. There were 10 calves in the control group and 10 calves in each of the experimental groups. All received the same feed but the experimental groups also received supplements of metal amino acid chelates shown in Table 9 below.

TABLE 9
Composition of Amino Acid Chelate Supplements fed to Calves

| | % in Mineral | % in Feed at Different Levels | | |
|---|---|---|---|---|
| Mineral | Mix Formula | 50 ppm | 100 ppm | 200 ppm |
| Iron | 9.00% | 0.0045% | 0.009% | 0.0180% |
| Copper | 2.15% | 0.00107% | 0.00215% | 0.0043% |
| Zinc | 3.00% | 0.0015% | 0.0030% | 0.0060% |
| Manganese | 1.20% | 0.0006% | 0.0012% | 0.0024% |
| Cobalt | 0.08% | 0.00004% | 0.00008% | 0.0002% |

The feed contained Cr₂O₃ as a marker. At 40 days and again at 80 days, 24-hour fecal samples were obtained from each animal and assayed for amino acid content, using the method described in Example 4. Knowing the metal amino acid content in the feed and the amount of undigested amino acids in the feeds following analysis, the data in Table 10 were developed.

TABLE 10
Percentage Increase of Available Amino Acids from Cattle Feeds in
Calves Receiving Amino Acid Chelates Compared to Control Calves

| Amino Acid | Newborn Calves % | Older Calves % |
|---|---|---|
| Aspartic Acid | +1.4 | +0.5 |
| Threonine | +3.6* | +2.1 |
| Serine | +7.5* | +3.9* |
| Glutamic Acid | +4.9* | .0 |
| Proline | +0.7 | −0.5 |
| Alanine | +4.7* | +4.4* |
| Valine | 0 | −0.2 |
| Methionine | +5.0* | +3.9* |
| Isoleucine | +0.3 | +1.5 |
| Leucine | +2.0 | −0.2 |
| Tyrosine | +5.1* | +3.7* |
| Phenylalanine | +1.8 | −0.2 |
| Lysine | +6.9* | +3.4* |
| Histidine | −1.7 | +0.6 |
| Arginine | +0.5 | 0 |

*p < 0.05

For almost every case when the amino acid chelates were included in the diet there were less amino acids in the feces. The animals metabolized more of the dietary protein in the feed.

While the invention has been described with reference to certain illustrative embodiments, those skilled in the art will recognize that numerous modifications, substitutions, and/or omissions can be made without departing from the spirit and scope of the present invention. It is therefore intended that the present invention be limited only by the appended claims.

What is claimed is:

1. A method of enhancing a mineral level in an animal with reduced environmental impact, comprising orally administering a metal amino acid chelate to an animal, wherein the amino acid to metal molar ratio of the metal amino acid chelate is from about 1:1 to 4:1, said metal contributing to a mineral level within the blood and tissues of the animal that is effective for stimulating growth of the animal to a greater degree than would be realized by administering the same amount of metal in the form of an inorganic metal salt, wherein the amount of the metal excreted in the feces of the animal is less than would be present when administering the same amount of metal in the form of the inorganic metal salt.

2. The method of claim 1, wherein the feces is substantially free of the metal due to the administration of the metal amino acid chelate, and wherein the feces would not be substantially free of the metal when administering the same amount of the metal in the form of the inorganic metal salt.

3. The method of claim 1, wherein the metal is a member selected from the group consisting of copper, zinc, manganese, calcium, magnesium, chromium, iron, and combinations thereof.

4. The method of claim 1, wherein the amino acid is a member selected from the group consisting of: alanine, arginine, aparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, valine, and dipeptides and tripeptides formed by any combination thereof.

5. The method of claim 1, wherein the metal amino acid chelate is coadministered with inorganic metal salts or vitamins.

6. The method of claim 1, wherein the metal amino acid chelate is coadministered with feed or is administered as a fortified feed.

7. The method of claim 1, wherein the metal amino acid chelate is coadministered with a liquid or is administered as a fortified liquid.

8. The method of claim 1, wherein the metal amino acid chelate is within a composition in particulate form.

9. The method of claim 1, wherein the animal is swine.

10. The method of claim 1, wherein the animal is ruminant.

11. The method of claim 1, wherein the animal is poultry.

12. The method of claim 1, wherein the animal is equine.

13. The method of claim 1, wherein the metal content of the feces in parts per million due to administering the metal amino acid chelate is reduced by 1 wt % to 70 wt % as compared to when the same amount of metal is administered in the form of the inorganic metal salt.

14. The method of claim 13, wherein the metal content of the feces in parts per million due to administering the metal amino acid chelate is reduced by 3 wt % to 50 wt % as compared to when the same amount of metal is administered in the form of the inorganic metal salt.

15. The method of claim 13, where the inorganic metal salt is selected from the group consisting of metal oxides or hydroxides, metal sulfates, metal chlorides, metal carbonates, and metal phosphates.

16. The method of claim 1, further comprising a preliminary step of determining an amount of metal needed to promote efficient growth of the animal.

17. The method of claim 16, wherein the step of administering includes delivering the amount of metal, while at the same time not substantially increasing the metal content of the feces compared to no metal administration.

18. The method of claim 1, wherein the amount of nitrates excreted in the feces of the animal is less than would be present if the animal ingested an amount by weight of amino acids from other dietary sources equal to that present in the metal amino acid chelate.

19. The method of claim 18, further comprising reducing the dietary protein fed to the animal from other dietary sources without reducing the rate of growth of the animal.

20. A method for facilitating the recovery of animal feces byproducts by reducing the amount of a metal in said feces byproducts, comprising:
orally administering a metal in the form of a metal amino acid chelate to an animal, wherein the amount of the metal administered is sufficient for stimulating growth of the animal while resulting in a lower amount of the metal in the feces of the animal than would result from administering the same amount of the metal in an inorganic salt form;
recovering the feces from the animal, said feces having a low enough concentration of metal to be acceptable for soil enrichment.

21. The method of claim 20, wherein the feces is substantially free of the metal.

22. The method of claim 20, wherein the metal is a member selected from the group consisting of copper, zinc, manganese, calcium, magnesium, chromium, iron, and combinations thereof.

23. The method of claim 20, wherein the amino acid is a member selected from the group consisting of: alanine, arginine, aparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, valine, and dipeptides and tripeptides formed by any combination thereof.

24. The method of claim 20, wherein the metal content of the feces in parts per million due to administering the metal amino acid chelate is reduced by 1% to 70% as compared to when the same amount of metal is administered in the form of an inorganic metal salt.

25. The method of claim 20, wherein the metal content of the feces in parts per million due to administering the metal amino acid chelate is reduced by 3% to 50% as compared to when the same amount of metal is administered in the form of an inorganic metal salt.

26. The method of claim 20, wherein the animal is swine.

27. The method of claim 20, wherein the animal is ruminant.

28. The method of claim 20, wherein the animal is poultry.

29. The method of claim 20, wherein the animal is equine.

30. The method of claim 20, wherein the amount of nitrates present in the feces is less than would be present if the animal had ingested an amount by weight of amino acids from other dietary sources equal to that present in the metal amino acid chelate.

31. The method of claim 20, further comprising the step of enriching soil with the feces after the recovering step.

32. A feed that provides a metal to a animal in a highly bioavailable form, comprising one or more metal amino acid chelates, where the amount of metal the animal ingests by eating the feed contributes to stimulating growth of the animal to a greater degree than the same amount of metal is ingested by the animal in the form of an inorganic metal salt, and where the amount of metal excreted in the feces of the animal is less than would be present when the same amount of metal is ingested by the animal in the form of the inorganic metal salt.

33. The feed of claim 32, wherein the metal is a member selected from the group consisting of copper, zinc, manganese, calcium, magnesium, chromium, iron, and combinations thereof.

34. The feed of claim 32, wherein the amino acid is a member selected from the group consisting of: alanine, arginine, aparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, valine, and dipeptides and tripeptides formed by any combination thereof.

35. The feed of claim 32, further comprising a foodstuff selected from the group consisting of corn, oats, soybeans, rice, wheat, barley, sorghum, canola, cottonseed meal, milk products, meat, bone meal, feather meal, food processing byproducts, and mixtures thereof.

36. The feed of claim 32, further comprising an additive selected from the group consisting of vitamins, flavor enhancers, aroma enhancers, colorings, fiber, yeast, ground limestone, potassium chloride, stabilizers, emulsifiers, sequestrants, preservatives, anti-oxidants, anti-caking agents, and mixtures thereof.

37. The feed of claim 32, wherein the feed is an admixture of the one or more metal amino acid chelates and a food.

38. The feed of claim 32, wherein the feed includes a food fortified with the one or more metal amino acid chelates.

39. The feed of claim 32, wherein the feed is a liquid feed which includes a solution or suspension of the one or more metal amino acid chelates.
